(19) United States
(12) Patent Application Publication
(10) Pub. No.: US 2020/0260955 A1
(43) Pub. Date: Aug. 20, 2020

(54) VIRTUAL ASSISTANT IN PULSE OXIMETER FOR PATIENT SURVEYS

(71) Applicant: KONINKLIJKE PHILIPS N.V., EINDHOVEN (NL)

(72) Inventors: John CRONIN, BONITA SPRINGS, FL (US); Michael D'ANDREA, BONITA SPRINGS, FL (US)

(21) Appl. No.: 15/776,188

(22) PCT Filed: Nov. 15, 2016
(86) PCT No.: PCT/EP2016/077675

§ 371 Date: May 15, 2018

Related U.S. Application Data
(60) Provisional application No. 62/259,052, filed on Nov. 23, 2015.

(30) Foreign Application Priority Data
Mar. 4, 2016 (EP) 16158628.4

(51) Int. Cl. A61B 5/00; A61B 5/1455; A61B 5/0205; G16H 40/63; G16H 40/67; G16H 80/00; G16H 10/20

(52) U.S. Cl. CPC A61B 5/0022; A61B 5/14551; A61B 5/0205; A61B 5/746; A61B 5/7435; G16H 10/20; A61B 5/749; G16H 40/63; G16H 40/67; G16H 80/00; A61B 5/741

(57) ABSTRACT

A method for monitoring a patient's condition using a patient questionnaire and a virtual assistant comprising: inputting one or more questions into a questions database; setting a threshold for at least one monitored pulse oximetry parameter; measuring at least one of blood oxygen saturation, pulse rate, and respiration rate via a pulse oximeter; displaying via a virtual assistant a questionnaire retrieved from the questions database when the threshold for the at least one monitored pulse oximetry parameter is exceeded; inputting an at least one answer to the one or more questions in the questionnaire; transmitting to a cloud server the acquired pulse oximeter data and the at least one answer to the one or more questions in the questionnaire; and accessing and displaying via the virtual assistant the acquired pulse oximeter data and the at least one answer to the one or more questions in the questionnaire.

### BACKGROUND OF THE INVENTION

Many medical conditions begin showing symptoms before they are fully diagnosed. Some of these medical conditions can be deadly if not given immediate treatment. For example, patients suffering from complications from cardiovascular diseases may exhibit fatigue, dizziness and shortness of breath. A study shows that more than 90% of heart attack victims survive if they reached the emergency room on time. The remaining 10% die because of major damage to the heart muscle. The mortality rate can be reduced if patients are given early or on-time treatment.

Using modern medical devices, the lag between symptoms detection and treatment can be reduced if not eliminated. A pulse oximeter can monitor blood oxygen saturation level, perfusion index, pulse rate and respiration rate. Thus, if a patient shows shortness of breath, this can be detected by a pulse oximeter, and the caregiver may be able to quickly assess if the symptoms shown are, for example, a possible prelude to a full-blown heart or asthma attack. Timely treatment can thus be provided to the patient before the patient's condition worsens.

However, a medical personnel cannot be present with a patient all the time to monitor a patient's condition should a patient exhibit some symptoms and a standard pulse oximeter detects them. Thus, it would be ideal to integrate a virtual assistant software to a patient monitor that would enable a quick self-assessment procedure, provide an initial diagnosis, and apply an appropriate response or action.

U.S. patent application 2013/0267795 discloses a health-monitoring system that prompts secondary health-related questions to the patient if a calculated score is within a suitable range. The system comprises a pulse oximeter as a measurement component. The system further comprises a web server, an interactive voice response system, a database of questions and results. WO patent publication 1998/050873 discloses a system that queries a user to provide additional information such as a self-assessment based on a pulse oximeter's measurement. The system transmits all the information to a central monitoring station.

### BRIEF SUMMARY OF THE INVENTION

The present invention relates to systems and methods for monitoring a patient's condition using a patient questionnaire and a virtual assistant. The system comprises a pulse oximeter connected to a patient monitor for monitoring one or more pulse oximetry parameter and for displaying the questionnaire. The questionnaire is displayed and answered via a virtual assistant. The system also comprises at least one database for storing the questions and answer of the patient, as well as the pulse oximeter data. Data on these databases can be accessed via a remote device. The method of the present invention comprises setting by a medical personnel a threshold for at least one monitored pulse oximetry parameter. When the set threshold is exceeded, a virtual assistant on the patient monitor will display a questionnaire for the patient to answer. The questionnaire includes questions set by the medical personnel, which can be edited and added locally or remotely through an interface. After the patient finishes answering the displayed questionnaire, the patient's answers are transmitted to and stored in a cloud server. The patient's answers can then be accessed locally or remotely via an authorized user or device.

### BRIEF DESCRIPTION OF THE DRAWINGS

The accompanying drawings, which are included to provide a further understanding of the invention, are incorporated herein to illustrate embodiments of the invention. Along with the description, they also serve to explain the principle of the invention. In the drawings:

FIG. 1 illustrates a block diagram of a system for monitoring a patient's condition using a patient questionnaire and a virtual assistant according to a preferred embodiment of the present invention.

FIG. 2 is a flowchart of a method for monitoring a patient's condition using a patient questionnaire and a virtual assistant according to a preferred embodiment of the present invention.

FIG. 3 is a flowchart of a method for setting thresholds according to a preferred embodiment of the present invention.

FIG. 4 is a flowchart of a method for monitoring a patient's condition using a patient questionnaire and a virtual assistant according to a preferred embodiment of the present invention.

FIG. 5 illustrates a preferred embodiment of a graphical user interface for setting thresholds.

FIG. 6 illustrates a preferred embodiment of a graphical user interface for adding and editing questions.

FIG. 7 illustrates a preferred embodiment of a graphical user interface of a patient monitor.

### DETAILED DESCRIPTION OF THE EMBODIMENTS

The following are definitions of terms as used in the various embodiments of the present invention.

The term “database” as used herein refers to a collection of data and information organized in such a way as to allow the data and information to be stored, retrieved, updated, and manipulated and to allow them to be presented into one or more formats such as in table form or to be grouped into text, numbers, images, and audio data. The database typically resides in computer memory that includes various types of volatile and non-volatile computer memory. “Database” as used herein also refers to conventional databases that may reside locally or that may be accessed from a remote location, e.g., remote network servers. The term “database” as used herein may also refer to a segment or portion of a larger database, which in this case forms a type of database within a database. Memory wherein the database resides may include high-speed random access memory or non-volatile memory such as magnetic disk storage devices, optical storage devices, and flash memory. Memory where the database resides may also comprise one or more software for processing and organizing data received by and stored into the database.

The term “questionnaire” as used herein refers to a collection of questions for use in a patient self-assessment and which can be customized based on the patient's medical condition.

The term “virtual assistant” as used herein refers to a computer-generated virtual assistant that guides the patient through the self-assessment process. The virtual assistant is able to display questions, process the patient's answers, and may optionally as well as provide feedback on the status of one or more medical devices in a patient's room.

The present invention relates to a method for monitoring a patient's condition using a patient questionnaire and a virtual assistant comprising: inputting one or more questions into a questions database; setting a threshold for at least one monitored pulse oximetry parameter; measuring at least one of blood oxygen saturation, pulse rate, and respiration rate via a pulse oximeter; displaying via a virtual assistant a questionnaire retrieved from the questions database when the threshold for the at least one monitored pulse oximetry parameter is exceeded; inputting an at least one answer to the one or more questions in the questionnaire; transmitting to a cloud server the acquired pulse oximeter data and the at least one answer to the one or more questions in the questionnaire; and accessing and displaying via the virtual assistant the acquired pulse oximeter data and the at least one answer to the one or more questions in the questionnaire.

The present invention also relates to a system for monitoring a patient's condition using a patient questionnaire and a virtual assistant comprising: a pulse oximeter; a questions and answers database for storing one or more questions and an at least one corresponding answer; a virtual assistant for displaying the one or more questions via a questionnaire and displaying the at least one corresponding answer, wherein the virtual assistant provides feedback relating to one or more pulse oximetry parameters and optionally one or more medical devices in the patient room; and a patient monitor connected to the pulse oximeter for monitoring one or more pulse oximetry parameters and for displaying the questionnaire via the virtual assistant when a set threshold for at least one monitored pulse oximetry parameters is exceeded.

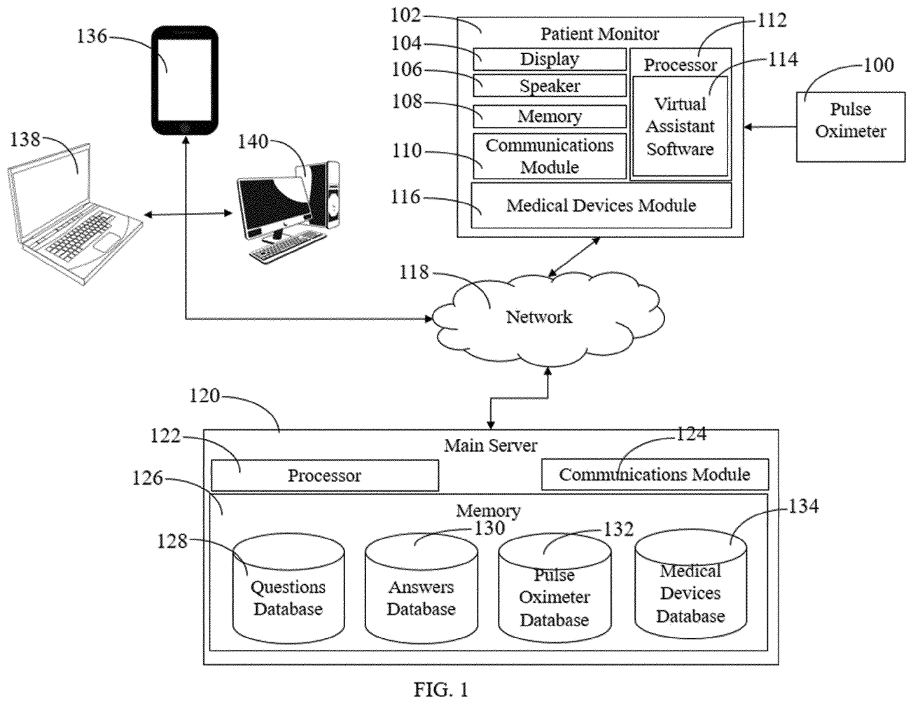

FIG. 1 illustrates a system for monitoring a patient's condition using a patient questionnaire and a virtual assistant according to a preferred embodiment of the invention. The system comprises a pulse oximeter 100 connected to a patient monitor 102, which in turn is connected to a network 118. The patient monitor 102 comprises a display 104, a speaker 106, a memory 108, a communications module 110, a processor 112, and a medical devices module 116. Also connected to the network is the server 120. The server comprises a processor 122, a communications module 124, and memory 126. The memory comprises a plurality of databases that includes a questions database 128, an answers database 130, a pulse oximeter database 132, and a medical devices database 134.

The pulse oximeter 100 preferably measures blood oxygen saturation, pulse rate and respiration rate. Blood oxygen saturation is a measure of the amount of oxygen carried by hemoglobin in the blood stream. It is usually expressed as a percentage rather than an absolute reading. For example, blood oxygen saturation levels measured immediately after birth can provide a good indicator of a baby's general state of health. Levels below 75% could indicate that the newborn infant may be suffering from some abnormality. To determine a patient's condition, the blood oxygen saturation should be expressed as a percentage of the total hemoglobin that is saturated with oxygen. Under many circumstances, that is the reading that pulse oximeters provides. Acceptable normal ranges for healthy patients range from 95 to 99 percent.

The pulse rate is the number of times the heart beats per minute. The pulse rate is characterized by the peaks in the pleth waveform. Acceptable normal pulse rates for healthy patients range from 60 to 100 beats per minute (bpm). For example, a pulse rate of 40 bpm could indicate a simple low blood pressure or bradycardia. On the other hand, a pulse rate of 100 bpm or more could indicate a fever or low blood sugar. When the pulse rate is irregular, it could indicate arrhythmia which could cause the heart not being able to pump enough blood to the body.

The respiration rate is the number of breaths per minute. The respiration rate is based on changes in the cardiovascular, respiratory and autonomic nervous systems that affects the pleth waveform. These changes can be used to calculate the respiration rate. Acceptable normal ranges for healthy patients range from 12 to 20 breaths/minute. Abnormal ranges of respiration rate may indicate asthma, pneumonia, congenital heart disease or drug overdose.

In a preferred embodiment of the present invention, the patient monitor 102 accesses either wirelessly or via wired connections one or more network server databases via the cloud/internet to retrieve and download the questionnaire into the patient monitor for use in the patient self-assessment. The patient monitor preferably comprises a controller for applying an action set by the medical personnel, one or more displays, a graphical unit interface for adding and editing questions and threshold values, an operating system, one or more processors, one or more input devices such as a keyboard or touchscreen display, one or more memory modules for storing data, and wireless or wired connectivity to other devices, such as tablets and cell phones, and to the cloud/internet. In accordance with the invention, the patient monitor preferably comprises a virtual assistant software 114 that guides the patient as the patient answers the questions in the questionnaire.

The virtual assistant integrated into the patient monitor preferably includes features and capabilities such as speech recognition, image matching, natural language processing, in addition to the question-and-answer system. Preferably, the virtual assistant receives input in the form of speech, as well as other input types such as camera-captured images. The virtual assistant's voice query also preferably uses a natural language processing question-and-answer system to produce a natural language response to the user. In a preferred embodiment of the present invention, the user's voice is preferably processed by a software based on automatic speech recognition that translates the user's speech into its text equivalent using statistical models. Using natural language processing methods, the question-and-answer feature preferably analyzes the patients inputted or selected answers to the questions and then implements a corresponding action based on the analysis of the patient's answers to the questions.

The virtual assistant software preferably comprises a voice synthesizer for reading aloud the questions and a selection of possible answers to the patient. The virtual assistant preferably also uses voice recognition to accept voice prompts for selecting an answer. For example, a patient feels dizzy because of low blood pressure. Thus, the patient is unable to read the questions and the presented selection of possible answers displayed on the screen. Preferably by enabling a, for example, text-to-voice feature, the virtual assistant can read aloud the questions and the presented selection of answers to the patient. Preferably, an option is provided that allows the questions and the selection of answers to be repeated or presented more than once. In the event that the patient is unable to speak such as when he or she is having difficulty breathing, the patient monitor preferably provides a built-in keyboard, touchpad or touch screen display for selecting or inputting an answer.

In accordance with a preferred embodiment of the invention, the virtual assistant software 114 preferably either transmits information to or receives information from one or more medical devices in the patient's room. These information are preferably transmitted to the server 120 and stored in the medical devices database 134. The information preferably can be accessed remotely only by authorized users and authorized devices. For example, one of the medical devices present in the patient room is an IV pump unit. The information fed to the IV pump unit may include the number of units constituting one dose/dosage form of one or more drugs to be administered to a patient; route of administration; frequency; duration if applicable (“for 5 days,” “until finished”); indication if applicable (e.g., “for infection” or “for blood pressure”), wherein these information are preferably transmitted to the server 120.

In one embodiment of the present invention, a patient is exhibiting some side effects from a drug the patient took and calls a designated attending physician. Upon receiving the patient's communication, the assigned medical personnel accesses various patient-related and treatment-related information stored in the medical devices database 134 via the physician's remote device such as a smart phone 136, laptop 138 or desktop computer 140. In this example, the attending physician accesses information relating to the one or more drugs recently administered to the patient, determines any known side-effects from the drug by accessing one or more medical databases, compares the patient's reaction to the drug with the drug's known side-effects, and determines if the proper dosage was administered to the patient at the proper time. In a preferred embodiment, the physician interacts with the virtual assistant to evaluate one or more patient-related information previously-inputted by the patient via the virtual assistant. The physician may, for example, determine via the virtual assistant whether the patient has inputted into the questionnaire any known allergies to one or more drugs and any additional information relating to the patient's family history. The physician may additionally query the virtual assistant regarding any known drug interactions between two or more drugs that the patient have taken during the last few hours or so.

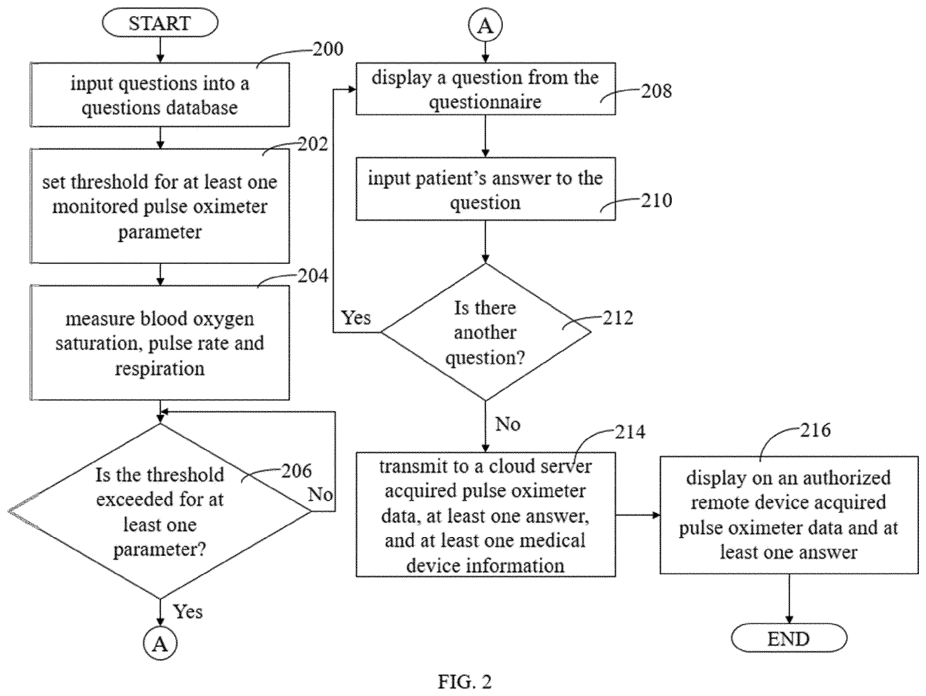

FIG. 2 illustrates a preferred method of the present invention. A medical personnel inputs questions into a questions database (step 200). The set of questions may be designed for a particular patient or a group of patients with a common medical condition. The medical personnel also sets a threshold for at least one pulse oximetry parameter (step 202). The pulse oximeter measures a patient's blood oxygen saturation, pulse rate and respiration rate (step 204). When the set threshold for at least one pulse oximetry parameter is exceeded (step 206), a virtual assistant displays a questionnaire on the patient monitor's display (step 208). The patient then inputs a corresponding answer (step 210), after which the virtual assistant displays the next question (step 212). When all the questions have been answered, the answers as well as the acquired pulse oximeter data and possibly other patient-related information obtained via the one or more medical devices present in the patient room are transmitted to a cloud server (step 214). The answers, pulse oximeter data, and other patient-related information collected using the one or more medical devices is stored into the answers database, pulse oximeter database, and medical devices database respectively. The answers, pulse oximeter data, and information from the medical devices can then be accessed through an authorized remote device by an authorized medical personnel (step 216).

The questions entered by a medical personnel is preferably stored in the database 128. The various sets of questions for different patients may be stored and organized in different ways, such as according to patient name or medical condition. Preferably, the set of questions can be retrieved or displayed according to a selected preferred mode of organization, such as according to latest or earliest date modified, patient name, associated one or more illnesses, patient status, date of patient admission into the hospital, and name of the attending medical personnel. In a highly preferred embodiment of the invention, the set of questions for a patient can be customized, retrieved, and selected from a database of prepared questions that correspond to different diseases. For example, a questionnaire for a patient initially diagnosed—by a different hospital or medical personnel, for example—to be suffering from a combination of two ailments or diseases may be customized by retrieving questions from a questions database that correspond to each of the two diseases and then combining them into a single set of questions that will be presented to the patient. For example, a patient initially diagnosed with a combination of pneumonia and asthma preferably will be presented with a customized set of questions that incorporates questions relating to both pneumonia and asthma. The customization of questions allows the attending medical personnel to more quickly narrow down a list of possible diagnosis that are consistent with the known patient symptoms and associated information. This minimizes the possibility of misdiagnosis and in turn allows the administration of prompt and appropriate treatment that leads to quicker patient recovery. An answers database 130 preferably stores the list of selectable answers for each corresponding question in the questionnaire. In a preferred embodiment, the questionnaire also allows the patient to input comments that describe in their own words, for example, how they feel at the moment. Preferably stored with each set of answers is a prescribed corresponding action to be performed by the patient, medical personnel, virtual assistant or patient monitor. Corresponding actions may include answering another question, performing the instructions provided by the virtual assistant or displayed on the patient monitor, or sending notifications to medical personnel, among others.

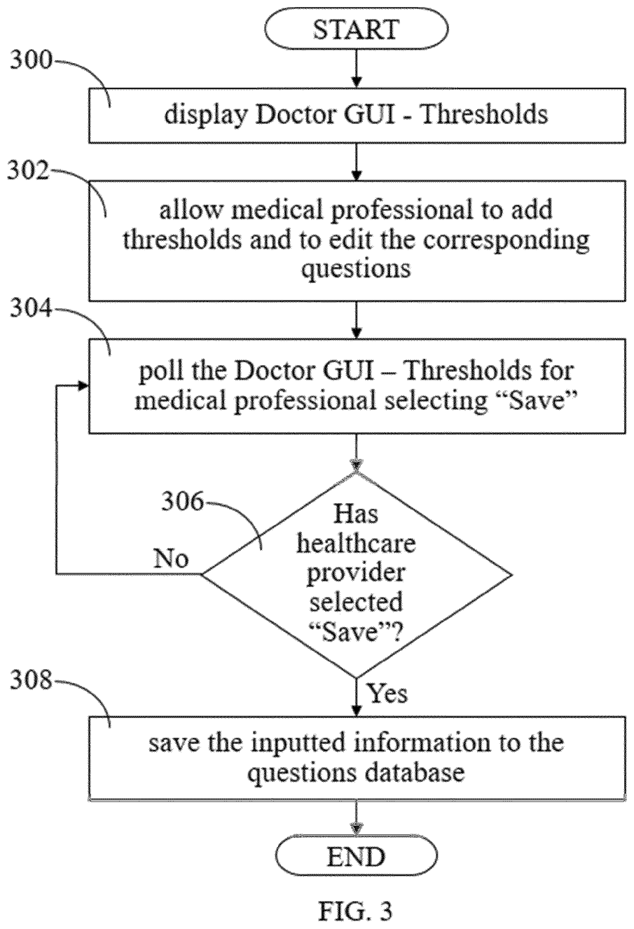

FIG. 3 illustrates a method for setting thresholds via a graphical user interface according to the present invention. In the first step of the process, the medical personnel's graphical user interface is enabled and displayed (step 300). Using the graphical user interface, the medical personnel sets the threshold values for at least one parameter such as a pulse oximetry parameter (step 302). Optionally, the medical personnel may also edit and update previously stored questions. The user interface is repeatedly polled until the medical personnel selects the “Save” option to update the threshold values and parameters monitored (step 304 and 306). Following this step, the updated thresholds are transmitted and stored into the questions database (step 308).

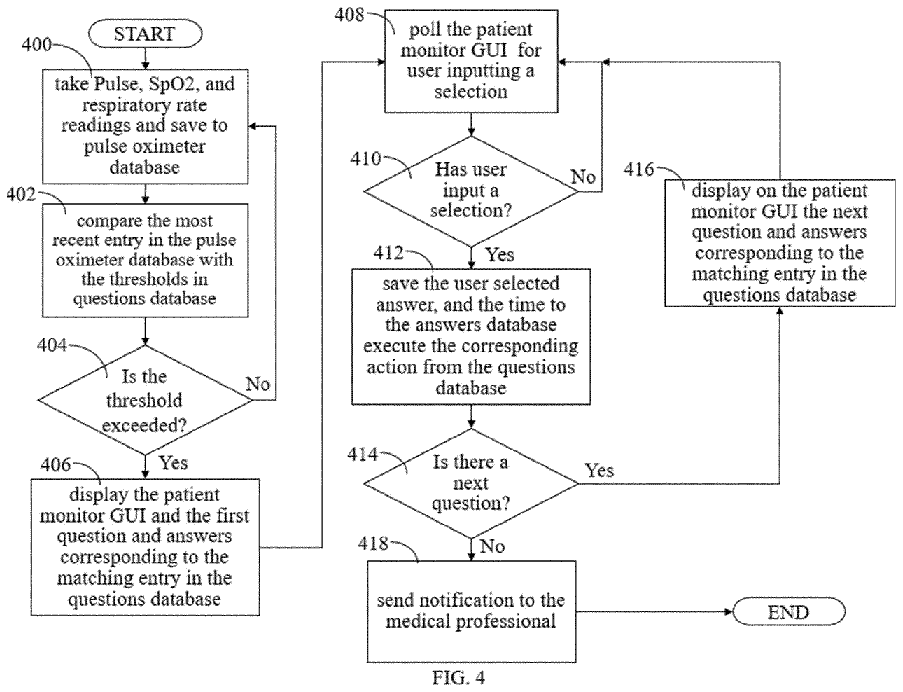

FIG. 4 illustrates a method involving a monitoring software according to the present invention. First, a patient's blood oxygen saturation, pulse rate and respiratory rate are measured, transmitted and stored to a pulse oximeter database (step 400). The measured pulse oximetry parameter are then each compared to a corresponding threshold (step 402). If at least one measured pulse oximetry parameter exceeds a threshold (step 404), a questionnaire is displayed on the patient monitor by a virtual assistant (step 406). The virtual assistant waits for a preset time until the patient has inputted an answer to at least one question (step 408). Once the user answers the question (step 410), the information is transmitted to and stored in the cloud (step 412). The information stored in the cloud comprises the patient's answer to the question and a timestamp that indicates the time the question was answered by the patient. The virtual assistant then determines if there is a next question (step 414). If there is, the virtual assistant displays the question and waits for the patient to input an answer (step 416). The process repeats until there are no more questions left. At this point, a notification will be sent to the medical personnel (step 418) who can then view and evaluate the patient's answers.

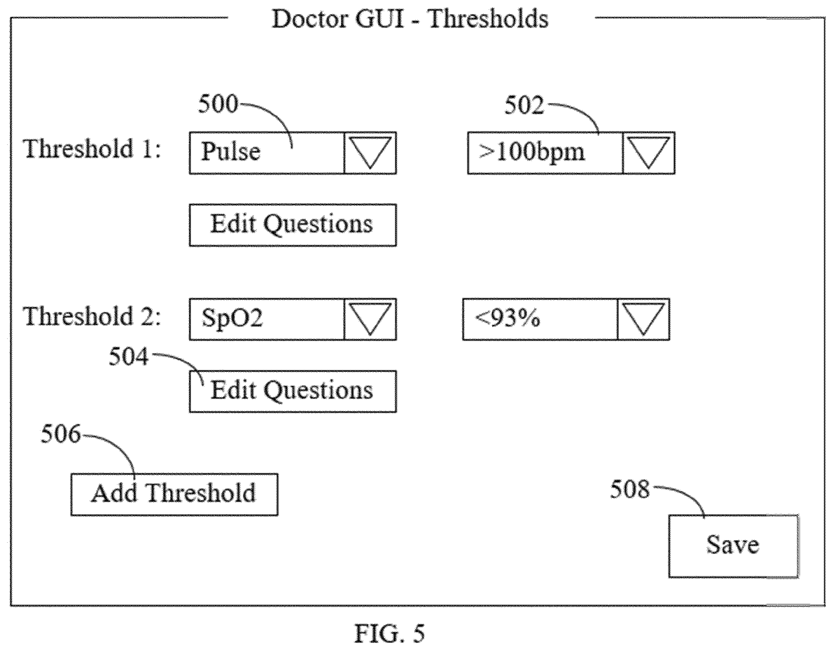

FIG. 5 illustrates a preferred embodiment of a graphical user interface window for editing and adding parameter thresholds. This GUI window comprises a first drop down menu 500 for choosing a pulse oximetry parameter and a second drop down menu 502 for selecting a corresponding parameter threshold range. The window further comprises a first button 504 to allow, for example, a medical personnel to edit the questions, a second button 506 for adding threshold values, and a third button 508 for saving updated threshold values. As shown in FIG. 5, “Pulse,” which refers to pulse rate, is selected from the drop down menu options as the parameter to be monitored, and “>100bpm” (greater than 100 beats per minute) is set as the pulse rate threshold range. Thus, if a measured pulse rate exceeds 100 bpm, the virtual assistant preferably displays a questionnaire on the patient monitor that present questions relating to the detected event, in this case, a patient's pulse rate that exceeds 100 bpm. In this example, the medical personnel has selected “SpO2” as a second pulse oximetry parameter to monitor, which here can be done by selecting the “Add Threshold” option. Also in this example, the medical personnel has selected “<93%” as the blood oxygen saturation's threshold range. Because there are two pulse oximetry parameters being monitored, different sets of questions may be displayed on the questionnaire depending on which pulse oximetry parameter exceeds the corresponding threshold range.

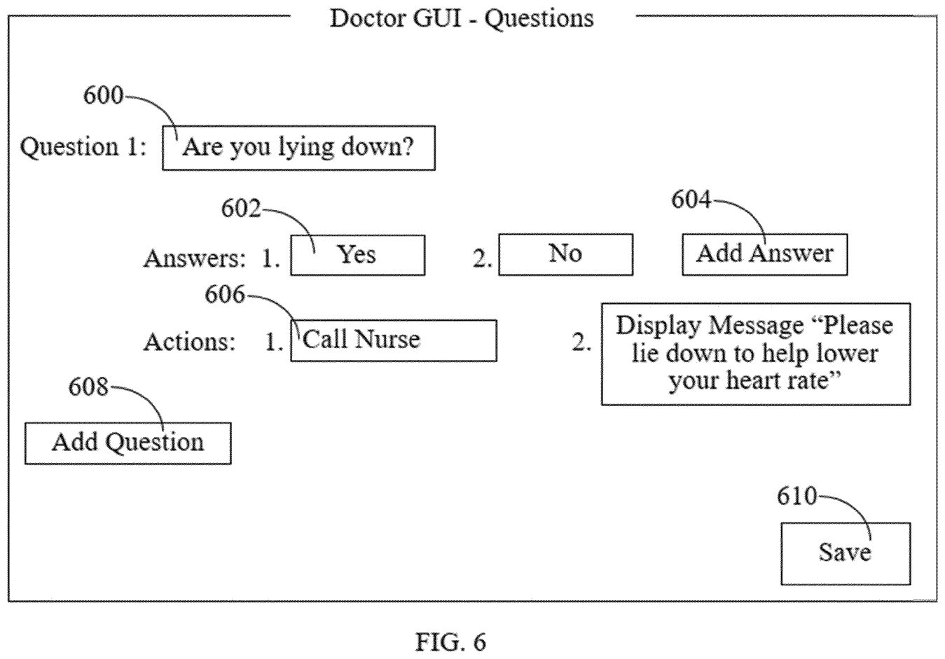

FIG. 6 illustrates a preferred embodiment of a graphical user interface window for editing and adding questions in a questionnaire. The GUI window for editing and adding questions is displayed when the button for editing questions 504 is selected from the window for editing and adding thresholds in FIG. 5. The GUI window used for editing and adding questions comprises a plurality of fields corresponding to a question 600 for the patient, corresponding answer to the question 602, and corresponding action 606 for the selected answer. The same GUI window further comprises a button “Add answer” 604 to allow the medical personnel to add answers, a button “Add question” 608 to allow the medical personnel to add questions and a button “Save” 610 to allow saving the updated questions and answers.

As shown in FIG. 6, a medical personnel inputs “Are you lying down?” in the field for Question 1. The medical personnel also inputs “Yes” and “No” as the selectable answers in the fields for Answer 1 and Answer 2 respectively. For each answer, the medical personnel also inputs a corresponding action to be performed by the virtual assistant. For example, if the selected answer is “Yes” the virtual assistant is instructed to “Call Nurse” by sending a notification to the nurses' station. If the selected answer is “No,” the virtual assistant is preferably programmed to display a message such as “Please lie down to help lower your heart rate” on the patient monitor.

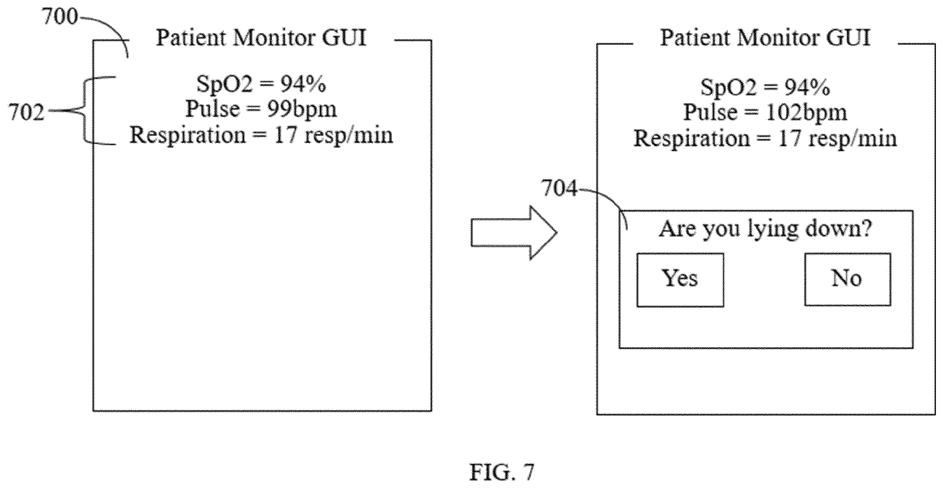

FIG. 7 illustrates another embodiment of the invention. Here, the patient monitor 102 displays a graphical user interface 700 that shows the monitored pulse oximetry parameters 702. On the left side of the figure, the patient monitor shows that the blood oxygen saturation (SpO₂) is at 94%, the pulse rate is at 99 bpm, and the respiration rate at 17 breaths per minute. After some time, the pulse rate is measured again and is found to have increased from 99 bpm to 102 bpm, a value that exceeds the preset threshold range. As a result of a detected exceeded parameter threshold range, the virtual assistant displays a question 704 through the graphical user interface that asks if the patient is currently lying down. This question is preferably followed by a series of other questions to determine a recommended course of action, e.g., instruct the virtual assistant to call an ambulance or doctor, or instruct the patient to simply lie down and rest.

In a preferred embodiment of the present invention, a doctor is remotely monitoring a patient with pneumonia and a history of asthma using a laptop. Using the laptop, the doctor adds questions and threshold values in the questions database for the patient self-assessment in case the patient shows symptoms of a possible asthma attack or if the patients exhibits symptoms due to complications from pneumonia. The doctor first adds a threshold value for the respiration rate, for example, greater than 18 breaths/min. The doctor then adds the first question to be displayed on the patient monitor if the threshold value is exceeded. The doctor inputs “Does your chest feel tight?” in the entry field for Question 1 on a GUI window. The doctor also inputs “Yes” and “No” as the selectable answers in the fields for Answer 1 and Answer 2, respectively. For each answer, the doctor also inputs a corresponding action to be performed by the virtual assistant. In this case, if the selected answer is “Yes” the virtual assistant is instructed to display another question such as “Do you hear wheezing sounds when you breathe?” If the patient-selected answer is “No,” the virtual assistant displays another question such as “Are you feeling dizzy?” on the patient monitor.

The doctor also inputs selectable patient answers and corresponding actions for each answer to each question. For example, the patient has a monitored respiration rate of 25 breaths/min, which exceeds the normal respiration rate and the respiration rate threshold previously set by a medical personnel. Upon detecting the exceeded threshold, the virtual assistant is automatically activated and accesses the questions database and displays and reads aloud the first question “Does your chest feel tight?” The patient answers “Yes,” and the virtual assistant then displays and reads aloud the second question “Are you hearing wheezing sounds when you breathe?” The question and answer session goes on until all the questions have been answered or until an action has been implemented by the virtual assistant. The patient's answers are preferably transmitted to the cloud server together with the previously acquired pulse oximeter data. Once the patient has completed answering all the questions, the patient monitor preferably sends a notification to the doctor's laptop notifying the doctor that the patient has finished answering the questionnaire. The doctor then remotely accesses the patient's answers and the patient's pulse oximeter data through the doctor's laptop via an internet connection to the one or more hospital databases. Based on the patient's answers and pulse oximeter data, the doctor diagnoses the patient as having an asthma attack. The doctor thus instructs the nearest available medical personnel to immediately attend to the patient to treat the patient's asthma.

In one aspect of the present invention, a patient is supposed to be resting after a heart bypass surgery. The pulse oximeter suddenly detects that the patient's pulse rate has exceeded the previously set threshold for the patient's pulse rate. The patient monitor's virtual assistant is activated and presents a questionnaire to the patient for self-assessment. The first question presented to the patient asks if the patient is lying down to determine if he is resting according to the attending physician's instructions. If the patient answers “Yes,” the virtual assistant sends a notification to the nurses' station via the patient monitor to attend to the patient because the patient could be suffering from an aftermath of the heart bypass surgery, e.g., tachycardia. If the patient answers “No,” the virtual assistant displays a recommended action on the patient monitor instructing the patient to lie down and rest to lower the patient's pulse rate. However, if the patient answers “no” but does not follow the one or more instructions based on the patient's unchanging measured pulse rate after a preset period, another action is preferably implemented by the virtual assistant such as sending a notification to an attending physician's device and the nurse's station desktop computer informing them about the situation.

In another embodiment of the present invention, an elderly patient in a nursing home experiences headache and dizziness. The pulse oximeter detects that the patient is experiencing low blood oxygen saturation level and respiration rate. This triggers the virtual assistant to display and read aloud the questionnaire to the patient for self-assessment. However, the elderly patient is also experiencing blurred vision, confusion and difficulty speaking and understanding, which are symptoms of a stroke. Thus, the patient is unable to respond to the virtual assistant and answer the questionnaire. The virtual assistant waits for a preset period of time, e.g., maximum of 10 seconds, until the question is answered. If the patient fails to respond after 10 seconds, the virtual assistant sends an alert to medical personnel in the medical facility to attend to the elderly patient.

The present invention is not intended to be restricted to the several exemplary embodiments of the invention described above. Other variations that may be envisioned by those skilled in the art are intended to fall within the disclosure.

1. A method for monitoring a patient's physiological condition using a patient questionnaire and a virtual assistant, the method comprising:
setting a threshold for at least one monitored pulse oximetry parameter of a patient;
measuring at least one of blood oxygen saturation, pulse rate, and respiration rate via a pulse oximeter;
storing a set of one or more questions in a questions database;
displaying on a screen of a patient monitor the set of one or more questions retrieved from the questions database when the threshold for the at least one monitored pulse oximetry parameter is met;
receiving at least one answer from the patient to the set of one or more questions;
transmitting to a cloud server the pulse oximeter data measurements and the at least one answer to the set of one or more questions;
retrieving the pulse oximeter data measurements and the at least one answer to the set of one or more questions from the cloud server to a medical professional's device; and
displaying a notification on the medical professional's device regarding a corresponding action based on the pulse oximeter data measurements and the at least one answer to the set of one or more questions; and
updating at least one question in the questions database based on input from an authorized source.

2. (canceled)

3. (canceled)

4. The method of claim 1, wherein at least one question is associated with a list of selectable answers.

5. The method of claim 4, wherein each answer is associated with one or more corresponding actions.

6. The method of claim 5, wherein the corresponding action in the notification is selected from the one or more corresponding actions.

7. The method of claim 1, wherein the displayed notification includes instructions to the patient.

8. A system for monitoring a patient's physiological condition using a patient questionnaire and a virtual assistant, the system comprising:
memory that stores a set threshold for at least one monitored pulse oximetry parameter of a patient;
a pulse oximeter that measures at least one of blood oxygen saturation, pulse rate, and respiration rate of the patient;
a questions and answers database that stores a set of one or more questions and at least one corresponding answer, the questions and answer database is updatable based on input from an authorized source;
a patient monitor screen for displaying the set of one or more questions retrieved from the questions and answers database when the threshold for the at least one monitored pulse oximetry parameter is met;
a patient interface that receives at least one answer from the patient to the set of one or more questions;
a communication interface that transmits to a cloud server the pulse oximeter data measurements and the at least one answer to the set of one or more questions,
wherein the patient monitor screen displays a notification regarding a corresponding action based on the pulse oximeter data measurements and the at least one answer to the set of one or more questions; and
wherein the notification is also displayed on a medical professional's device when the pulse oximeter data measurements and the at least one answer to the set of one or more questions are retrieved from the cloud server.

9. (canceled)

10. (canceled)

11. The system of claim 8, wherein at least one question is associated with a list of selectable answers.

12. The system of claim 11, wherein each answer is associated with one or more corresponding actions.

13. The system of claim 12, wherein the corresponding action in the notification is selected from the one or more corresponding actions.

14. The system of claim 8, wherein the communication interface sends the notification over a communication network to a designated recipient.

15. A non-transitory computer-readable storage medium, having embodied thereon a program executable to perform the method of claim 1.
